(19) United States
(12) Patent Application Publication
(10) Pub. No.: US 2025/0280775 A1
(43) Pub. Date: Sep. 11, 2025

(54) CONTROL SYSTEM

(71) Applicant: Provectus IP Pty Ltd, Noosaville (AU)

(72) Inventor: Nusqe SPANTON, Queensland (AU)

(21) Appl. No.: 19/218,536

(22) Filed: May 26, 2025

Related U.S. Application Data
(63) Continuation of application No. 17/287,570, filed on Apr. 22, 2021.

(30) Foreign Application Priority Data
Oct. 22, 2018 (AU) 2018903996

(51) Int. Cl. A01G 33/00; A01G 7/04; H05B 45/10; H05B 45/20; H05B 47/105

(52) U.S. Cl. CPC A01G 33/00; A01G 7/045; H05B 45/10; H05B 45/20; H05B 47/105

(57) ABSTRACT

A control system for growing plant matter such as algae, the control system having a controller for controlling a plurality of LED lights, wherein the controller is adapted to control the spectrum and intensity of the LED lights. Preferably the controller is also adapted to control a flickering frequency of the LED lights.

### CROSS-REFERENCE TO RELATED APPLICATIONS

This application is a continuation-in-part of U.S. application Ser. No. 17/287,570, filed Apr. 22, 2021, which is a National Stage filing of PCT/AU2019/051152, filed Oct. 22, 2019, each of which is hereby incorporated by reference in its entirety.

### FIELD

The present invention relates to a control system for growing algae. Reference will be made in the specification to the use of the invention with respect to growing algae. The patent specification describes this use, but it is by way of example only and the invention is not limited to this use.

### BACKGROUND

Commercial cultivation of Algae is used to produce food ingredients, food colorants and dyes, bioplastics, pharmaceuticals, and algal fuel amongst others. Most Algae requires water, nutrients, a carbon source and light to grow.

How all these elements are administered determines the quality and quantity of the algae. There are a large number of combinations of how these elements can be administered to grow different types of algae.

Light is an important element in growing algae. Direct sunlight is too strong for most algae. However, direct sunlight is often best for strong growth as the algae underneath the surface is able to utilize the less intense light created from the shade above.

One prior art solution to control the exposure of algae to light is to agitate the algae.

### SUMMARY

It is an object of the present invention to overcome or at least alleviate one or more of the above mentioned problems with growing algae and/or provide the consumer with a useful or commercial choice.

In one aspect, the present invention broadly resides in a control system for growing plant matter having

a controller for controlling a plurality of LED lights, wherein the controller is adapted to control the spectrum and intensity of the LED lights.

Preferably, the controller is adapted to control a flickering frequency of the LED lights. Preferably the flickering frequency is a frequency of on/off cycles of the LED light. Preferably the controller is adapted to control the flickering frequency in a range of 2 ms to 5,000 ms. More preferably the controller is adapted to control the flickering frequency in a range of 25 ms to 1,000 ms. In another embodiment, the controller is adapted to control the flickering frequency in a range of 2 ms to 500 ms. In a further embodiment, the controller is adapted to control the flickering frequency in a range of 10 ms to 100 ms.

Preferably the controller includes a communication module. The communication module is preferably adapted to communicate with a remote device. In one embodiment, the remote device is a mobile device such as a mobile telephone. In another embodiment, the remote device is a computer. In a further embodiment, the remote device is a server. Preferably the controller communicates with the server via the internet.

In one embodiment, the controller is adapted to communicate with more than one remote device.

The remote device is preferably adapted to set the spectrum and intensity of the LED lights. The remote device is preferably adapted to set the flickering frequency of the LED lights.

Preferably the controller is adapted to communicate with one or more sensors. Preferably the one or more sensors provide data on the growth of the plant matter.

Preferably the one or more sensors include one or more gas sensors. Preferably the one or more gas sensors include a carbon dioxide, hydrogen and/or oxygen sensor. In one embodiment, the one or more gas sensors are adapted to sense dissolved gas in growing medium used to grow plant matter such as algae. The growing medium is preferably a liquid such as water.

Preferably the one or more sensors include a sensor to determine plant growth. Preferably the one or more sensors includes a spectrophotometer to determine plant growth. In one embodiment, the one or more sensors includes a spectrophotometer to determine algae cell density in liquid culture.

Preferably the one or more sensors include a temperature sensor. Preferably the temperature sensor senses a temperature of the growing medium.

Preferably the one or more sensors includes a pH sensor to measure the pH of the growing medium.

Preferably the one or more sensors include a salinity sensor. Preferably the salinity sensor measures the salinity of the growing medium.

Preferably the one or more sensors include a nitrate sensor. Preferably the nitrate sensor measures the concentration of nitrates in the growing medium.

Preferably the one or more sensors include a cell count sensor. Preferably the cell count sensor measures the quantity of plant matter cells in a defined volume.

Preferably the one or more sensors include a cell health sensor. Preferably the cell health sensor provides data on the health of plant matter cells.

The controller is preferably adapted to receive data from the one or more sensors. The controller is preferably adapted to send the data from the one or more sensors to the remote device. The controller is preferably adapted to send data on the spectrum and intensity of the LED lights to the remote device. The controller is preferably adapted to send data on the flickering frequency of the LED lights to the remote device. The remote device is preferably adapted to optimise the spectrum and intensity of the LED lights based on the data from the one or more sensors. Preferably the remote device correlates data from the one or more sensors with data on the spectrum and intensity of the LED lights. Preferably the remote device correlates data from the one or more sensors with data on the flickering frequency of the LED lights. Preferably the remote device will determine the spectrum and intensity of the LED lights that correspond to desirable data from the one or more sensors. Preferably the remote device will set the spectrum and intensity values in the controller to the determined spectrum and intensity of the LED lights. Preferably the remote device will determine the flickering frequency of the LED lights that corresponds to desirable data from the one or more sensors. Preferably the remote device will set the flickering frequency in the controller to the determined flickering frequency of the LED lights.

Preferably the remote device will correlate the type of plant matter being grown with the data on the spectrum and intensity of the LED lights. Preferably the remote device will correlate the type of plant matter being grown with the data from the one or more sensors. Preferably the remote device will correlate the type of plant matter being grown with the data on the flickering frequency of the LED lights.

In one embodiment, a remote device defines the type of plant matter being cultivated and communicates this data with the controller, the controller communicates this data with a further remote device and downloads from the further remote device spectrum and intensity settings of the LED lights which correlate with the type of plant matter being cultivated.

Preferably the remote device will correlate desired traits of plant matter being grown with the data on the spectrum and intensity of the LED lights. Preferably the remote device will correlate desired traits of plant matter being grown with the data from the one or more sensors. Preferably the remote device will correlate desired traits of plant matter being grown with the data on the flickering frequency of the LED lights.

In one embodiment, a remote device defines the desired traits of plant matter being cultivated and communicates this data with the controller, the controller communicates this data with a further remote device and downloads from the further remote device spectrum and intensity settings of the LED lights which correlate with the desired traits of plant matter being cultivated.

In another embodiment, the remote device is used to manually set the spectrum and intensity settings of the LED lights in the controller.

Preferably the controller is adapted to control the spectrum of the LED lights in the range of 200 nm to 800 nm. More preferably the controller is adapted to control the spectrum of the LED lights in the range of 380 nm to 750 nm.

Preferably the LED lights to provide the plant matter with light.

Preferably the controller is adapted to control each of the plurality of LED lights individually. In one embodiment the plurality of LED lights are divided into zones, with the controller adapted to control each zone individually. In one embodiment, each zone is used to cultivate different plant matter. In another embodiment, each zone is used to cultivate plant matter at a different growth stage. In a further embodiment, each zone is used to develop different traits in the plant matter.

Preferably the plant matter is algae.

Preferably the control system includes the LED lights.

Preferably the control system includes the one or more sensors.

In one embodiment, the control system includes the remote device.

In another aspect, the present invention broadly resides in an algae cultivation control system, the control system having a controller for controlling a plurality of LED lights to provide the algae with light, wherein the controller is adapted to control the spectrum and intensity of the LED lights.

In a further aspect, the present invention broadly resides in a control system for growing algae having a controller for controlling a plurality of LED lights, wherein the controller is adapted to control the spectrum and intensity of the LED lights.

In another aspect, the present invention broadly resides in in a control system for growing algae having a plurality of LED lights to provide the algae with light; and a controller for controlling the plurality of LED lights, wherein the controller is adapted to control the spectrum and intensity of the LED lights.

Preferably the control system includes one or more sensors. Preferably the one or more sensors provide data on the growth of the algae.

Preferably the control system includes a communication module. More preferably, the controller includes the communication module. The communication module is preferably adapted to communicate with a remote device. The communication module is preferably adapted to enable communication between the remote device and the controller.

In one embodiment, the remote device is adapted to communicate with one or more further control systems. Preferably the remote device is adapted to communicate the settings for the spectrum and intensity of the LED lights which have resulted in desirable traits in the plant matter to the one or more further control systems.

Preferably the remote device is adapted to communicate additional settings to the control system and or the one or more further control systems. Preferably the additional settings include one or more of flow rate of growing medium, temperature of growing medium, amount of CO₂ added, amount of O₂ added, and or the type and or amount of nutrients added. Preferably the corresponding controller is adapted to control one or more pumps, valves, solenoids, heaters, or the like, to control the flow rate of growing medium, temperature of growing medium, amount of CO₂ added, amount of O₂ added, and or the type and or amount of nutrients added. Preferably the corresponding controller controls one or more pumps, valves, solenoids, heaters, or the like, to control the flow rate of growing medium, temperature of growing medium, amount of CO₂ added, amount of O₂ added, and or the type and or amount of nutrients added, according to the settings received from the remote device.

In a further aspect, the present invention broadly resides in a method of controlling the growing of algae, the method including the steps of controlling with a first control system the spectrum and intensity of a plurality of LED lights for growing algae; monitoring with one or more sensors traits of the algae; changing the spectrum and intensity of the plurality of LED lights while continuing to monitor with the one or more sensors traits of the algae; correlating traits of the algae with the spectrum and intensity of the plurality of LED lights; transmitting with a remote device the spectrum and intensity settings of the plurality of LED lights for a desired trait of the algae to a second control system to grow algae with the desired traits using the second control system.

Preferably the method includes the step of transmitting from the first control system to the remote device the spectrum and intensity settings of the plurality of LED lights and the correlating sensor data for the traits of the algae. Preferably the step of correlating traits of the algae with the spectrum and intensity of the plurality of LED lights is performed on the remote device.

Preferably the method further includes correlating traits of the algae with the flow rate of growing medium, temperature of growing medium, amount of CO₂ added, amount of O₂ added, and or the type and or amount of nutrients added.

Preferably the method includes changing one or more of the spectrum and or intensity settings of the plurality of LED lights, flow rate of growing medium, temperature of growing medium, amount of CO₂ added, amount of O₂ added, and or the type and or amount of nutrients added to optimise the traits of the algae. Preferably AI is used to change one or more of the spectrum and or intensity settings of the plurality of LED lights, flow rate of growing medium, temperature of growing medium, amount of CO₂ added, amount of O₂ added, and or the type and or amount of nutrients added to optimise the traits of the algae. Preferably the method further includes transmitting with the remote device settings related to one or more of flow rate of growing medium, temperature of growing medium, amount of CO₂ added, amount of O₂ added, and or the type and or amount of nutrients added to the second control system to grow algae with the desired traits using the second control system.

In another aspect, the present invention broadly resides in a method of controlling the growing of algae, the method including the steps of controlling with a first control system the spectrum and intensity of a plurality of LED lights for growing algae; monitoring with one or more sensors traits of the algae; changing the spectrum and intensity of the plurality of LED lights while continuing to monitor with the one or more sensors traits of the algae; correlating traits of the algae with the spectrum and intensity of the plurality of LED lights; controlling the spectrum and intensity settings of the plurality of LED lights to correspond to a desired trait of the algae.

It will be appreciated that traits of the algae can include cell count, growth rate, cell health, algae cell density and or the like.

The features described with respect to one aspect also apply where applicable to all other aspects of the invention. Furthermore, different combinations of described features are herein described and claimed even when not expressly stated. For example the features described in relation to the control system for growing plant matter can apply to the algae cultivation control system and the control system for growing algae and vice versa.

### BRIEF DESCRIPTION OF THE DRAWINGS

In order that the present invention can be more readily understood reference will now be made to the accompanying drawings which illustrate a preferred embodiment of the invention and wherein:

FIG. 1 is a schematic view of a control system according to an embodiment of the present invention;

FIG. 2 is a schematic view of a portion of a control system according to another embodiment of the present invention; and

FIG. 3 is a schematic view of a portion of a control system according to a further embodiment of the present invention.

### DETAILED DESCRIPTION

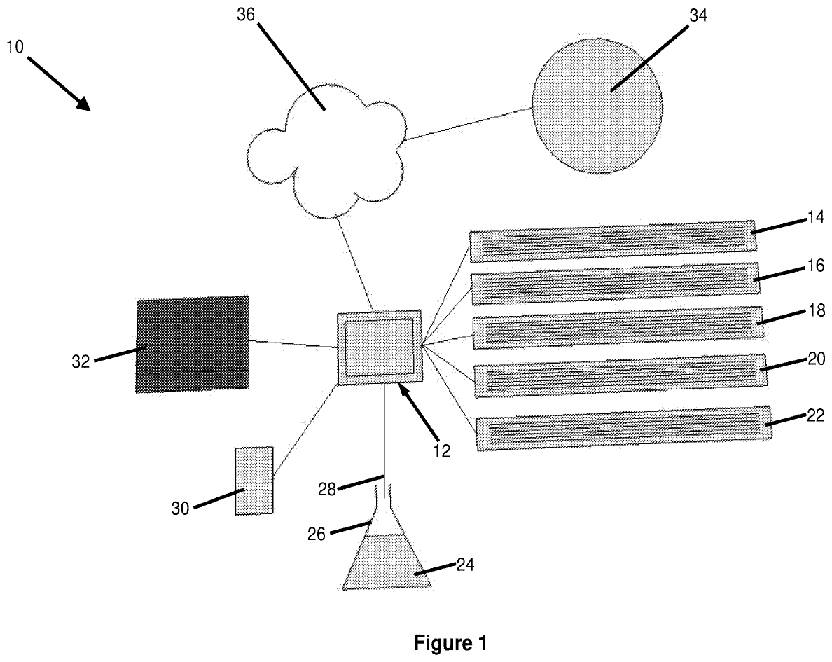

With reference to FIG. 1 there is shown a control system according to an embodiment of the present invention in the form of an algae cultivation control system 10. The control system 10 includes a controller 12.

The controller 12 controls a plurality of LED lights 14,16,18,20,22. The controller 12 is adapted to control the spectrum of the LED lights 14,16,18,20,22. The controller 12 is adapted to control the intensity of the LED lights 14,16,18,20,22. The controller 12 is adapted to control the flickering frequency of the LED lights 14,16,18,20,22.

The LED lights 14,16,18,20,22 provide light to plant matter in the form of algae 24 being grown/cultivated, in a growing medium in the form of water, in a container 26.

One or more sensors 28 monitor the growth of the algae and communicate the data to the controller 12.

The controller 12 communicates with remote devices in the form of a mobile device 30, a computer 32 and a server 34. The controller 12 communicates with the server 34 via the internet 36.

The controller 12 is able to communicate the settings of the LED lights 14,16,18,20,22 and the data from the one or more sensors 28 to the mobile device 30, computer 32 and server 34. The settings of the LED lights 14,16,18,20,22 in the controller 12 can be set by the mobile device 30, the computer 32 and/or the server 34.

The server 34 can correlate data from the one or more sensors 28 with settings of the spectrum, the intensity and/or the flickering frequency of the LED lights 14,16,18,20,22. The server 34 can determine the spectrum, the intensity and/or the flickering frequency of the LED lights 14,16,18,20,22 that correspond to desirable data from the one or more sensors 28. The server 34 can set the spectrum, intensity and/or the flickering frequency values in the controller 12 to the determined values. The mobile device 30, the computer 32 and/or the server 34 can set which data from the one or more sensors 28 is desirable.

The server 34 can correlate data for different types of algae being grown and/or different traits of the algae 24 being grown. The mobile device 30, the computer 32 and/or the server 34 can set the different types of algae being grown, or the traits of the algae being grown.

The controller 12 can control the spectrum, the intensity and/or the flickering frequency of the LED lights 14,16,18,20,22 individually. In this manner, LED lights 14,16,18,20,22 can define different zones, in which different algae is being grown and/or in which different traits of algae are being cultivated.

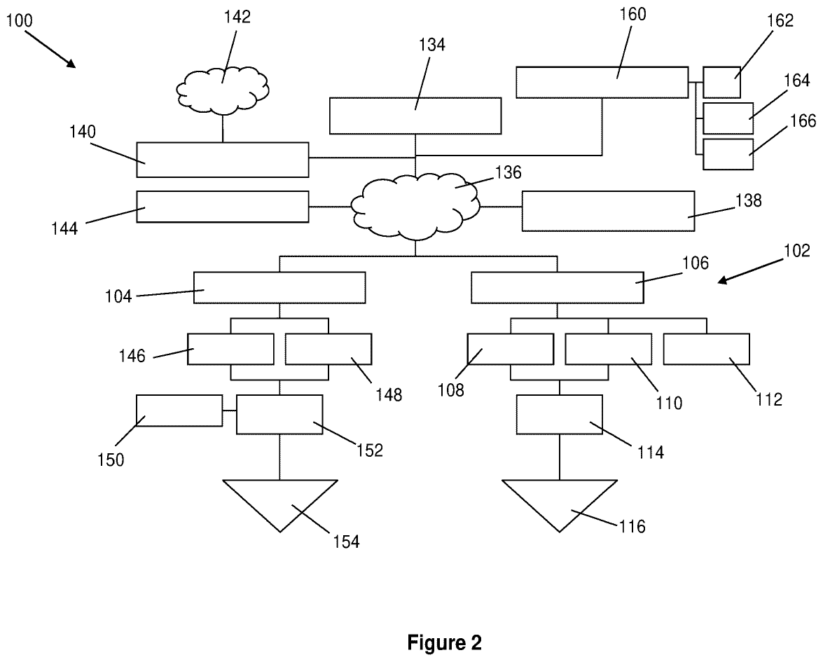

With reference to FIG. 2, there is shown an algae cultivation control system 100. The algae cultivation control system 100 includes a first control system in the form of a pilot control system 102, and a second control system in the form of a user control system 104.

The pilot control system 102 includes a controller 106 for controlling the spectrum, intensity and flickering rate settings of the plurality of LED lights, flow rate of growing medium, temperature of growing medium, amount of CO₂ added, amount of O₂ added, and or the type and or amount of nutrients added in the cultivation of algae at 108 and 110. The controller 106 also monitors sensors (not shown) which provide data on the growth of the algae at 108 and 110. The controller 106 also monitors the waste products created at 112. At 114 the grown algae is processed and at 116 the end product is dispatched. The pilot plant is used to test the effects of different settings of the spectrum, intensity and flickering rate settings of the plurality of LED lights, flow rate of growing medium, temperature of growing medium, amount of CO₂ added, amount of O₂ added, and or the type and or amount of nutrients added in the cultivation of algae at 108 and 110.

The data from the sensors and the settings of the spectrum, intensity and flickering rate settings of the plurality of LED lights, flow rate of growing medium, temperature of growing medium, amount of CO₂ added, amount of O₂ added, and or the type and or amount of nutrients added is sent via the internet 136 to a remote device in the form of a server 134.

The server 134 processes the data and settings in an optimisation module 140. The optimisation module 140 uses artificial intelligence 142 to optimise the settings to achieve desired traits of the algae. The server 134 maintains a database of optimised settings for different algae strains and different desired traits. On request through a pilot user console 138, the server 134 can communicate the updated settings to the pilot control system 102.

If a user is growing a known strain of algae using the user control system 104, they can via a user console 144, download optimised settings from the server 134 to the user control system 104, to control the cultivation of algae at 146 and 148. The user control system also monitors the waste production at 150. At 152 the grown algae is processed and at 154 the end product is dispatched.

Data and settings from the user control system 104 is uploaded via the internet 136 to the server 134. The data and settings are fed into the optimisation module 140 so that the artificial intelligence 142 can use the data and settings to further optimise the settings to achieve desired traits of the algae.

The server 134 includes a notification module 160 which can notify users and pilot users (not shown) via Short Message Service (SMS) 162, email 164 or AV 166 to new algae recipes or optimizations, or alert users if settings or data are outside of a predetermined range for the pilot control system 102 or user control system 104 respectively.

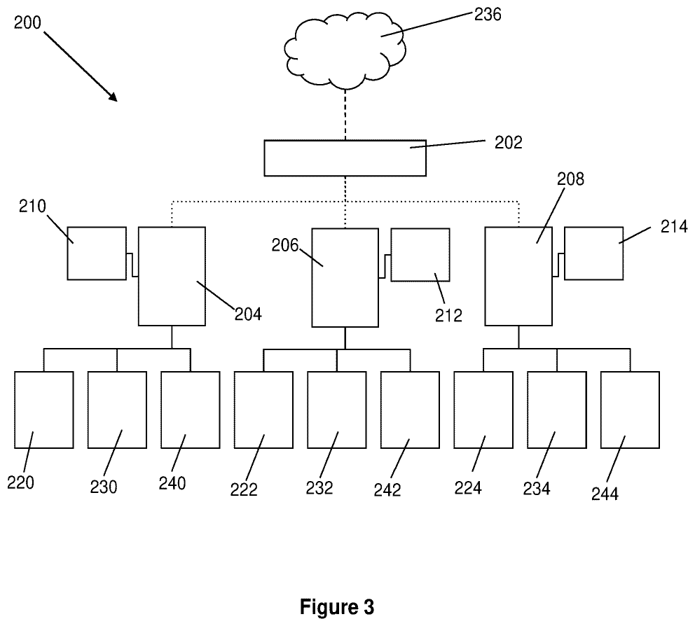

With reference to FIG. 3, there is shown a control system 200. The control system 200 has a controller 202 which controls multiple sections of an algae cultivation system in the form of an inoculation section 204, a grow section 206 and an inoculation section 208.

Each of the sections 204, 206, 208 has a localized controller 210,212,214. The localized controllers 210,212,214 each control LED lights 220,222,224 respectively. The localized controllers 210,212,214 each control solenoids and pumps 230,232,234 respectively to control the flow rate of growing medium, amount of CO₂ added, amount of O₂ added, and or the type and or amount of nutrients added. The localized controllers 210,212,214 also monitor sensors 240,242,244. The controller 202 can send setting and sensor data to a remote server (not shown) via the internet 236 via Internet of Things (IoT) messaging system. The controller can also receive IoT messages to control the LED lights 220,222,224 and the solenoids and pumps 230,232,234 via the respective localized controllers 210,212,214.

An advantage of the preferred embodiment of the control system includes the ability to control the growth of different types of plant matter. Another advantage of the preferred embodiment of the control system includes the ability to control different traits of plant matter being grown. A further advantage of the preferred embodiment of the control system includes that the controller can set the spectrum, intensity and/or the flickering frequency values of the LED lights in response to desired data values from the one or more sensors.

While the foregoing has been given by way of illustrative example of this invention, all such and other modifications and variations thereto as would be apparent to persons skilled in the art are deemed to fall within the broad scope and ambit of this invention as is herein set forth.

Throughout the description and claims of this specification the word “comprise” and variations of that word such as “comprises” and “comprising”, are not intended to exclude other additives, components, integers or steps.

1. A method of controlling the growing of algae in a liquid growing medium, the method comprising:
controlling, by a first control system, a spectrum and an intensity of a plurality of light emitting diode (LED) lights for growing algae;
monitoring, by one or more sensors, traits of the algae;
changing the spectrum and the intensity of the plurality of LED lights while performing the monitoring;
correlating, on a remote device, the traits of the algae with the spectrum and the intensity of the plurality of LED lights; and
transmitting, by the remote device, spectrum and intensity settings of the plurality of LED lights for a pre determined trait in the traits of the algae to a second control system, to grow the algae with the pre-determined trait using the second control system,
wherein:
the first control system comprises a pilot control system for testing effects of different settings in the spectrum and intensity settings on the traits of the algae;
the one or more sensors comprises: a spectrophotometer to determine growth or cell density of the algae in the liquid growing medium, and/or a cell count sensor configured to measure a quantity of cells of the algae in a pre-determined volume of the liquid growing medium;
the remote device comprises a computer server, the computer server comprising an optimizer for optimizing, by artificial intelligence, the spectrum and intensity settings based on data produced by the one or more sensors, to achieve the pre-determined trait; and
the second control system comprises a user control system for growing the algae with the pre-determined trait based on the optimized spectrum and intensity settings for achieving the pre-determined trait.

2. The method of claim 1, wherein the pre-determined trait is selected from the group consisting of: increased cell count, increased growth rate, increased cell health, increased algae cell density, and combinations thereof.

3. The method of claim 1, wherein the artificial intelligence further optimizes LED light spectrum and intensity settings in the second control system, to achieve the pre-determined trait.

4. The method of claim 3, wherein the first control system is further configured to test one or more effects of one or more parameters on the traits of the algae, wherein the one or more parameters is selected from the group consisting of: different flickering rate settings of the plurality of LED lights, a flow rate of the liquid growing medium, a temperature of the liquid growing medium, an amount of carbon dioxide (CO₂) added to the liquid growing medium, an amount of (O₂) added to the liquid growing medium, a type of nutrients added in the liquid growing medium, an amount of nutrients added to the liquid growing medium, and combinations thereof.

5. The method of claim 4, wherein the optimizer is further configured to process data corresponding to the one or more parameters, and wherein the artificial intelligence optimizes the spectrum and intensity settings based on the data corresponding to the one or more parameters to achieve the pre-determined trait.

6. The method of claim 1, wherein the computer server maintains a database of different optimized spectrum and intensity settings for different strains of the algae and different traits in the traits of the algae.

7. The method of claim 1, wherein the computer server comprises a notifier configured to notify users of either the first control system or the second control system of any new settings in the optimized spectrum and intensity settings.

8. The method of claim 1, wherein the computer server comprises a notifier configured to alert users of either the first control system or the second control system when any of the spectrum and intensity settings or any of the data produced by the one or more sensors are outside of a pre-determined range.

9. The method of claim 1, further comprising:
downloading, by a user console, the optimized spectrum and intensity settings from the computer server to the second control system, to control the growing of the algae with the pre-determined trait.

10. The method of claim 1, wherein the one or more sensors further comprises: a temperature sensor for sensing a temperature of the liquid growing medium; a gas sensor for sensing dissolved gas in the liquid growing medium; a sensor for determining growth of the algae; a pH sensor for measuring pH of the liquid growing medium; a salinity sensor for measuring salinity of the liquid growing medium; a nitrate sensor for measuring a concentration of nitrates in the liquid growing medium; and/or a cell health sensor for measuring health of one or more cells of the algae.

11. The method of claim 1, wherein each of the first and the second control systems comprises a controller, wherein the controller of the first control system is configured to control the spectrum and intensity settings of the plurality of LED lights, and wherein the controller of the second control system is configured to control spectrum and intensity settings of a second set of LED lights.

12. The method of claim 11, wherein each of the controllers is configured to control one or more features selected from the group consisting of: a flow rate of the liquid growing medium, a temperature of the liquid growing medium, an amount of carbon dioxide (CO₂) added to the liquid growing medium, an amount of oxygen (O₂) added to the liquid growing medium, a type of nutrients added in the liquid growing medium, an amount of nutrients added to the liquid growing medium, and combinations thereof.

13. The method of claim 12, wherein each of the controllers is configured to monitor the data produced by the one or more sensors.

14. The method of claim 13, wherein each of the controllers is configured to:
receive the spectrum and intensity settings of the plurality of LED lights transmitted from the remote device, and
control, based on the received spectrum and intensity settings, (i) one or more pumps, one or more valves, one or more solenoids, and/or one or more heaters, and (ii) the one or more features.
